(19) United States
(12) Patent Application Publication
(10) Pub. No.: US 2004/0101533 A1
(43) Pub. Date: May 27, 2004

(54) Medicament for preventing or treating tumors caused by human papilloma virus type 18

(76) Inventors: Rainer Muller, Munchen (DE); John Nieland, Stockdorf (DE); Josef Gabelsberger, Munchen (DE); Ruth Herbst, Gilching (DE)

Correspondence Address: FOLEY AND LARDNER, SUITE 500, 3000 K STREET NW, WASHINGTON, DC, 20007, US

(21) Appl. No.: 10/433,091

(22) PCT Filed: Nov. 30, 2001
(86) PCT No.: PCT/EP01/14038

(30) Foreign Application Priority Data
Dec. 1, 2000 (DE) 100 59 630.4

(51) Int. Cl. A61K 39/12

(52) U.S. Cl. 424/186.1

(57) ABSTRACT

A medicament for the prevention or treatment of human papillomavirus type 18 (HPV-18)-specific tumor comprising at least one fusion protein of at least one L1 protein and at least one E protein of one or more HPV-18 and, where appropriate, suitable additives and/or excipients, characterized in that the fusion protein is an L1ΔCE7_{x-y} fusion protein, where x is an integer from 1 to 3 inclusive and y is an integer from 61 to 64, in particular 62 to 64, especially 62 or 64.

The present invention relates to a medicament for the prevention or treatment of human papillomavirus type 18 (HPV-18)-specific tumor comprising at least fusion protein of at least one L protein and at least one E protein of one or more HPV-18 and, where appropriate, suitable additives and/or excipients, where the fusion protein is a L1ΔCE7_{x-y} fusion protein where x is an integer from 1 to 3 inclusive and y is an integer from 61 to 64, in particular 62 to 64, especially 62 or 64.

Papillomaviruses, also called wart viruses, are double-stranded DNA viruses having a genome size of about 8 000 base pairs and an icosahedral capsid having a diameter of about 55 nm. To date, more than 100 different human papillomavirus types have been disclosed, some of which, e.g. HPV-16, HPV-18, HPV-31, HPV-33, HPV-39, HPV-45, HPV-52 or HPV-58, may cause malignant tumors, and others, e.g. HPV-6, HPV-11 or HPV-42, may cause benign tumors.

Electron microscopic analyses of BPV-1 and HPV-1 revealed that the viruses are composed of 72 pentameric capsomeres which in turn consist of five L1 molecules (Baker, T. et al. (1991) Biophys. J., 60, 1445).

The genome of papillomaviruses can be divided into three regions: the first region relates to a noncoding region which comprises regulatory elements for transcription and replication of the virus. The second region, called the E (early) region, comprises various protein-encoding segments E1-E7, of which, for example, the E6 protein and the E7 protein is responsible for the transformation of epithelial cells, and the E1 protein controls the DNA copy number. The E6 region and E7 region are so-called oncogenes which are also expressed in cells showing malignant degeneration. The third region, also called the L (late) region, comprises two protein-encoding segments L1 and L2 which code for structural components of the viral capsid. The L1 protein is more than 90% present in the viral capsid, with the L1:L2 ratio generally being 30:1.

HPV-6 and HPV-11 are thought to be responsible inter alia for genital warts, and some papillomavirus types such as HPV-16, HPV-18, HPV-31, HPV-33, HPV-39, HPV-45, HPV-52 and HPV-58 are associated with malignant tumors of the anogenital tract. In about 10%-20% of cases, HPV-18 is thought to be associated with cancer of the cervix (cervical carcinoma). HPV-18 is therefore a potent risk factor for the development of cervical neoplasias. In addition, the immune system plays an important part in the progression of the disease. Thus, presumably cellular immune responses and, in particular, antigen-specific T lymphocytes are important for the defense mechanism. It has further been found that the E7 gene is constitutively expressed in all layers of the infected epithelium in high-grade cervical intraepithelial neuroplasias (CIN II/III) and cervical tumor. The E7 protein is therefore regarded as a potential tumor antigen and as target molecule for activated T cells (see, for example, WO 93/20844). The E7-induced cellular immune response in patients is, however, apparently insufficiently strong to influence the progress of the disease. The immune response may possibly be enhanced by suitable vaccines.

It has now been possible to show that expression of the L1 gene and coexpression of the L1 gene and L2 gene forms virus-like particles (VLPs). It was possible to use the VLPs to produce neutralizing antibodies in various animal systems. However, the production of virus-neutralizing antibodies is of little clinical significance if the viral infection has already taken place, because a virus-specific cytotoxic T-cell (CTL) appears to be necessary to eliminate virus-infected cells. This is why so-called chimeric papillomavirus-like particles (CVLPs) consisting of a chimeric HPV-16 L1-E7 protein have been developed (Müller, M. et al. (1997) Virology, 234, 93): some CVLPs induce an E7-specific CTL response in mice, although experiments on inducing antibodies by immunization of mice with CVLPs against E7 failed (Müller, M. et al. (1997), supra). In addition, neutralizing antibodies of HPV-associated diseases in patients appeared to limit the immune response to administered L1 protein (Müller, M. et al. (1997), supra). However, CVLPs are still of interest for the development of a vaccine because E7 proteins of tumor cells presented by class I MHC molecules would represent target molecules of CTLs.

Peng et al. (1998) Virology, 240, 147 has now described CVLPs consisting of C-terminally truncated L1 of bovine papillomavirus (BPV) and HPV-16E7₄₉₋₅₇, which induce E7-specific cytotoxic T cells, and protect from the growth of E7-expressing tumors, after immunization of C57B1/6 mice. Greenstone et al. (1998) Proc. Natl. Acad. Sci. USA, 95, 1800 describe CVLPs which consist of HPV-16L1 plus HPV-16L2 fused to the full-length HPV-16E7 protein and which, after immunization of C57Bl/6 mice, protect from the growth of epithelial E7-expressing tumor cells, although cytotoxic T cells were not detected and thus induction of the immune response appears to be less efficient.

The production of VLPs and CVLPs generally takes place by genetic manipulation through expression of the corresponding genes coding for one or more L proteins or L and E proteins in suitable expression systems. The corresponding genes are described for example by Kirnbaum, R. et al. (1994) J. Virol., 67, 6929-6936 and obtainable through the EMBL database. The access numbers are, for example, for HPV18: PAPHPV18; for HPV31: PAPPPH31; for HPV33: PAPPPH33 or for HPV58: PAPPPH58.

Suitable expression systems are, for example, genetically manipulated yeasts, e.g. Saccharomyces (cerevisiae), Pichia (pastoris), Kluyveromyces (lactis), Schizosaccharomyces (pombe) or Hansenula (polymorpha) (Carter, J. J. et al. (1991), Virology, 182, 513), insect cells such as, for example, Trichoplusia ni high five and Spodoptera frugiperda Sf9 and SF+ (see, Müller et al. (1997), supra) or prokaryotic cells (see, for example, WO 96/11272). When the particles are produced in prokaryotic cells, they are generally precipitated in the cell and form so-called inclusion bodies which must be subsequently renatured and dissolved. For use of the particles or capsids or precursors thereof, the so-called capsomeres, produced by genetic manipulation, further purification steps are necessary after expression.

A crucial disadvantage of the anti-HPV active substances described in the literature is, however, that, firstly, they show only a small effect and, secondly, to date it has not been possible to show any effective immunotherapy of HPV-18-specific tumor.

It was therefore an object of the present invention to provide a medicament with which human papillomavirus-specific tumor can be effectively prevented or treated, which can be produced simply and which appears suitable for approval as medicament.

It has now been found, surprisingly, that the medicament of the invention is suitable for inducing in in vivo and in in vitro test systems cellular immune responses to HPV18 fusion proteins so that these medicaments of the invention are also effective against HPV-18-specific tumor.

One aspect of the present invention is therefore a medicament for the prevention or treatment of human papillomavirus type 18 (HPV-18)-specific tumor comprising at least one fusion protein of at least one L protein and at least one E protein of one or more HPV-18 and, where appropriate, suitable additives and/or excipients, where the fusion protein is an L1ΔCE7_{x-y} fusion protein, where x is an integer from 1 to 3 inclusive and y is an integer from 61 to 64, in particular 62 to 64, especially 62 or 64. The fusion protein is preferably a CVLP.

This fusion protein preferably comprises no papillomavirus-nonspecific epitopes or papillomavirus-nonspecific amino acid sequences.

Papillomavirus-nonspecific epitopes mean for the purposes of the present invention generally epitopes in the fusion protein which are caused by a foreign protein content, by post-translational modifications or by misfolding of the papillomavirus-specific proteins. A foreign protein content means amino acid sequences which are not attributable to papillomavirus-specific proteins.

The papillomavirus-nonspecific epitopes may be a cause for example of it not being possible to prevent or control effectively a papillomavirus-specific tumor although neutralizing antibodies or CTL immune responses are induced, because the immunological effect is diminished by nonspecific antibodies or CTLs, or immunological side effects interfere with the effect of the actual active substance.

A further preferred embodiment is a medicament for the prevention or treatment of human papillomavirus type 18 (HPV-18)-specific tumor comprising at least one fusion protein of at least one L protein and at least one E protein of one or more HPV-18 and, where appropriate, suitable additives and/or excipients, where the fusion protein is an L1ΔCE7_{x-y} fusion protein, where x is an integer from 1 to 3 inclusive and y is an integer from 61 to 64, in particular 62 to 64, especially 62 or 64.

The fusion protein preferably comprises at least one papillomavirus-nonspecific epitope comprises. This may be located for example at the particular point of fusion between the L proteins, the E proteins or an L protein and an E protein. This epitope may moreover be encoded by an HPV18- or HPV-foreign nucleic acid, where an HPV18 or HPV-foreign nucleic acid means a nucleic acid which is not to be found in the natural genome of an HPV18 or of an HPV. Since such epitopes cannot be generated by HPV18-infected cells of a patient who has possibly become more or less tolerant of the HPV18 infection, such an additional epitope, especially in the form of a cytotoxic T-cell epitope, may lead to breaking through the immunotolerance of the HPV18 L1 and E proteins. Effects of this type are described for example by Lehmann PV et al. (1993, Immuno. Today 14(5), 203-8), in which an immune response depends on a first immunogenic peptide.

The medicament of the invention preferably acts to prevent or treat benign or malignant tumor, especially laryngeal, cervical, penile, vulval or anal carcinoma, including precursors thereof, such as, for example, high-grade CIN (cervical intraepithelial neoplasia).

In a further preferred embodiment, the medicament of the invention comprises no adjuvant, e.g. no substance which enhances the immunity of the papillomavirus-specific protein because, in particular, the presence of an L protein, especially of L1, itself sufficiently enhances the immunity. This property is advantageous in particular for the approval as medicament or diagnostic aid, because the only immunostimulating materials approved by the approval authorities at present are aluminum salts. In addition, the omission of adjuvants and/or other excipients or additives prevents unwanted side effects.

As already mentioned above, a further essential problem in the use of capsids and capsomeres as medicaments is their poor solubility. Thus, for example, capsids or capsomeres of HPV-18 are prone to aggregation, thus considerably reducing the solubility. The solubility of the capsids and capsomeres, which is low in some cases, leads not only to a loss of yield but also to difficulty in use as medicaments.

In a further preferred embodiment, the medicament of the invention therefore comprises as a suitable additive or excipient about 0.1 to about 3 M, preferably about 0.15 to about 1 M, in particular about 0.2M of a salt having a pH of about 7 to about 8, preferably of about 7.3 to 7.4, in particular of 7.4.

The advantage of this salt solution is that the fusion protein remains in solution as capsid or capsomere, or is finely dispersed as suspension. The salt is generally an alkali metal or alkaline earth metal salt, preferably a halide or phosphate, in particular an alkali metal halide, especially NaCl and/or KCl. The use of NaCl is particularly preferred for producing a pharmaceutical formulation.

The pH of the medicament is generally adjusted using a suitable organic or inorganic buffer such as, for example, preferably a phosphate buffer, Tris buffer (tris (hydroxymethyl)aminomethane), HEPES buffer ([4-(2-hydroxyethyl)piperazino]ethanesulfonic acid) or MOPS buffer (3-morpholino-1-propanesulfonic acid). The selection of the particular buffer generally depends on the desired buffer molarity. Phosphate buffer is suitable for example for solutions for injection and infusion.

Examples of further additives and/or excipients suitable for example for further stabilization of the papillomavirus-specific protein in the medicament of the invention are detergents such as, for example, polyoxyehtylene sorbitan fatty acid esters (polysorbates) such as, for example, polysorbate 80 (for example Tween 80®), polysorbate 60 (for example Tween 60®) or polysorbate 20 (for example Tween 20® polyoxyethylene alkyl ethers (for example Brij 58® Brij 35®) or others such as, for example, Triton X-100®, Triton X-114®, NP40 ®, Span 85, Pluronic 121 or sodium deoxycholate. Further suitable are also polyols such as, for example, polyethylene glycol or glycerol, sugars such as, for example, sucrose or glucose, zwitterionic compounds such as, for example, amino acids such as glycine or, in particular, taurine or betaine and/or a protein, such as, for example, bovine or human serum albumin. Detergents, polyols and/or zwitterionic compounds are preferred. Other additives and/or excipients are protease inhibitors such as, for example, aprotinin, 68 -aminocaproic acid or pepstatin A. Preferred additives are those which do not induce immunological side effects.

For the purposes of the present invention, the terms L protein, L1 protein, L2 protein, L1/L2 protein and E protein mean both the full-length proteins and mutants thereof such as, for example, deletion mutants.

In a further preferred embodiment, the fusion protein of the invention comprises a deleted L protein, preferably a deleted L1 protein and, where appropriate, L2 protein. The deletion has the advantage that other proteins, for example papillomavirus-specific E protein sequence, can be particularly effectively inserted into the deleted region, thus permitting the range of application of the composition of the invention to be extended. Particular preference is given to an L protein with a C-terminal deletion and, in particular, a C-terminally deleted L1 protein. The C-terminal deletion has the advantage that the efficiency of the production of virus-like particles can be increased because the nuclear localization signal located at the C terminus is deleted. The C-terminal deletion therefore preferably comprises up to about 35 amino acids, preferably about 15 to about 35 amino acids, especially about 26 to 28 amino acids.

In a further preferred embodiment, the E protein is also deleted, especially the E6 and/or E7 protein. It is preferred in particular for the C-terminal part of the E protein to be deleted, preferably the C-terminal part of the E7 protein, because these constructs are able in conjunction with deleted L protein to form preferably capsomeres and/or capsids. Particular preference is given to deletions of up to 55 amino acids, preferably about 5 to about 55 amino acids, in particular about 40 to about 51 amino acids, especially about 41 to about 45 amino acids. A further preferred embodiment is when the N terminus of an E protein is deleted in addition or as alternative to a C-terminal deletion, preferably the N-terminal part of the E7 protein, because constructs of this type permit packaging of more C-terminal amino acids into capsids.

It emerged in the construction of HPV16 L1_{ΔC} fusion proteins that the capsid-formation ability is lost depending on the length and the specific sequence of the fusion partner fused to HPV16 L1_{ΔC} (Müller, M. et al. 1997 supra and DE 19812941). Thus, capsid formation is reduced in HPV16 L1_{ΔC*}E7₁₋₆₀ compared with HPV16 L1_{ΔC*}E7₁₋₅₅ and is completely prevented in HPV16 L1_{ΔC*}E7₁₋₆₅. The reason for this is likely to be that sequences in the region of amino acids 55 to 70 of HPV16 interfere with capsid formation. It is possible that the cysteines present in this region form incorrect disulfide bridges with one another or with cysteines of the L1 portion. At the same time, however, the E protein partner fused to L1_{ΔC} should comprise as many epitopes as possible for activation of cytotoxic T cells.

It has now surprisingly been found for HPV18 that, in contrast to HPV16, even constructs with an E7 portion of more than 55 amino acids form capsids.

It was possible to establish, equally surprisingly, particularly in view of the analogy to HPV16, that capsid formation in these HPV18 constructs was not prevented by the presence of a cysteine in the C-terminal region either. Thus, it was possible to show for example that the construct HPV18 L1_{ΔC*}E7₁₋₆₄, which contains one of these cysteines, forms capsids just as efficiently as the considerably shorter construct HPV18 L1_{ΔC*}E7₁₋₅₅.

It has also surprisingly been found that HPV18 L1_{ΔC*}E7 constructs starting with the methionine at amino acid position 1 of E7 have the advantage compared with constructs having an N-terminal deletion of E7, for example of the methionine at position 1, that they comprise a mouse T-cell epitope which starts with the methionine at position 1 (present in peptides Q43 and Q44 from example 5).

This epitope is surprising because it is not predicted by the so-called peptide prediction program of Parker et al. (1994), J. Immunol. 152:163, under http://www-bimas.dcrt.nih/gov/molbio/hla_bind/.

These HPV18 constructs thus have the advantage that their induction of a cellular immune response can be checked very simply in appropriate mouse strains, because immunization of these mice induces a specific T-cell response which is detectable using the peptides Q43 and/or Q44. Without such an immunological test which is simple to carry out it would be necessary to guess from the content of the capsids the immunological activity thereof, which undoubtedly leads to less accurate results.

It has additionally been possible to demonstrate, surprisingly, that the HPV18 L1_{ΔC*}E7₁₋₆₄ construct induces in the mouse model a cytotoxic immune response, whereas the HPV18 L1_{ΔC*}E7₂₋₆₂ construct does not. It can be concluded from this that the construct HPV18 L1_{ΔC*}E7₁₋₆₄ is more immunogenic than the construct HPV18 L1_{ΔC*}E7₂₋₆₂, so that a far greater probability of success exists for the HPV18 L1_{ΔC*}E7₁₋₆₄ construct in human vaccinations too.

Preferred constructs are HPV18 L1_{ΔCDI}E7_{1-53DI} and HPV18 L1_{ΔCDI}E7_{1-60DI}. In these constructs, the E7 portion is flanked at the DNA level for constructional reasons by two EcORV restriction cleavage sites. This means that the fusion protein contains the additional amino acids Asp and Ile (DI) in front of and behind the E7 portion.

Further preferred constructs are HPV18 L1_{ΔC*}E7₂₋₅₇, HPV18 L1_{ΔC*}E7₂₋₆₀, HPV18 L1_{ΔC*}E7₂₋₆₂ and HPV18 L1_{ΔC*}E7₃₋₆₄. These constructs marked with an * contain no HPV-foreign sequences.

Particularly preferred constructs are HPV18 L1_{ΔC*}E7₁₋₅₅, HPV18 L1_{ΔC*}E7₁₋₅₇, HPV18 L1_{ΔC*}E7₁₋₆₂ and HPV18 L1_{ΔC*}E7₁₋₆₄These constructs marked with an * contain no HPV-foreign sequences.

The present invention therefore also relates to the use of the constructs of the invention for producing a medicament on the one hand for the prevention of HPV-specific tumors, and on the other hand for the regression of already existing HPV-specific tumors.

To produce a medicament having both prophylactic and therapeutic efficacy, it is preferred for the described papillomavirus-specific fusion protein to be in the form of a capsid and/or capsomere, because the immune response can be markedly increased further by the capsids and/or capsomeres and, in particular, by the content of L protein. Preferred fusion proteins suitable for capsid and/or capsomere formation are therefore, for example, fusion proteins of deleted L1 and E7, E6 and/or E1.

Capsids are for the purposes of the present invention viral or virus-like structures in a generally icosahedral form which are generally composed of 72 capsomeres.

Capsomeres are for the purposes of the present invention assembled proteins comprising at least one papillomavirus structural protein, preferably L1 or deletions of L1. For example, 5 fusion proteins of the invention may assemble to form one capsomere which are in turn able to assemble to form one capsid.

To produce a combination vaccine it is advantageous to combine proteins or peptides from different HPV types, preferably from HPV-6, HPV-11, HPV-16, HPV-18, HPV-31, HPV-33, HPV-35, HPV39, HPV-45, HPV-52 and/or HPV-58, for example a combination of HPV-16 and HPV-18 or HPV 18, HPV-31, HPV-45 and HPV-58 in the case of, for example, cervical carcinoma or HPV6 and HPV-11 in the case of, for example, condylomas.

A further aspect of the present invention is a method for producing a medicament of the invention, in which a suitable cell comprising a suitable expression vector which codes for said fusion protein is cultivated under suitable conditions, the expression product is isolated, and suitable additives and/or excipients are added where appropriate.

The expression vectors may be, for example, prokaryotic or eukaryotic expression vectors. Examples of prokaryotic expression vectors for expression in E. coli are, for example, the vectors pGEM or pUC derivatives (see, for example, WO 96/11272). Examples of eukaryotic expression vectors for expression in Saccharomyces cerevisiae are, for example, the vectors p426Met25 or p426GAL1 (Mumberg et al. (1994) Nucl. Acids Res., 22, 5767-5768, Carter, J. J. et al. (1991) supra) and for expression in insect cells are, for example, baculovirus vectors, especially the Autographa Californica virus as disclosed in EP-B1-0 127 839 or EP-B1-0 549 721 (see, for example, also WO 94/20137), and for expression in mammalian cells are, for example, the vectors Rc/CMV and Rc/RSV or SV40 vectors, which are all generally available. However, commercially available baculovirus expression systems are also suitable, such as, for example, the Baculo Gold™ transfection kit from Pharmingen or the Bac-to-Bac™ baculovirus expression systems from Gibco BRL. Further suitable expression systems are recombinant vaccinia viruses (see, for example, WO 93/02184).

In general, the expression vectors also comprise promoters suitable for the particular host cell, such as, for example, the trp promoter for expression in E. coli (see, for example, EP-B1-0 154 133), the ADH2 promoter for expression in yeast (Russel et al. (1983), J. Biol. Chem. 258, 2674-2682), the baculovirus polyhedrin promoter for expression in insect cells (see, for example, EP-B1-0 127 839 or U.S. Pat. No. 5,004,687) or the early SV40 promoter or LTR promoters for example of MMTV (mouse mammary tumor virus; Lee et al. (1981) Nature 214, 228-232).

Examples of suitable host cells are the E. coli strains DH5, HB101 or BL21, the Saccharomyces, Pichia, Kluyveromyces, Schizosaccharomyces or Hansenula yeast strains (Carter, J. J. et al. (1991), Virology, 182, 513), insect cell lines of the genus Lepidoptera, e.g. of Spodoptera frugiperda, Trichoplusia ni, Rachiplusia ou or Galleria mellonela or the animal cells COS, C127, Vero, 293 and HeLa, all of which are generally available (see, for example, WO 94/00152).

The coding nucleic acids for the individual papillomavirus-specific proteins have been isolated and cloned for example via PCR (polymerase chain reaction) amplification from a gene library. The genome of HPV18 is generally available under the GenBank accession No. X05015 and was published by Cole and Danos (J. Mol. Biol. 1987, 193 (4), 599-608).

The sequence used as basis for constructing the fusion proteins of the invention had the following alterations in the L1 gene for this purpose: at the DNA level a C was exchanged for a G at positions 89, 848, 1013 and 1230 of the L1 gene. At the protein level, the first three alterations lead to an exchange of Pro by Arg, where the last mutation results in no alteration at the protein level.

The DNA sequence of the HPV18L1_{ΔC*} used as basis is thus:

atggctttgtggcggcctagtgacaataccgtatatcttccacctccttc
tgtggcaagagttgtaaataccgatgattatgtgactcgcacaagcatat
tttatcatgctggcagctctagattattaactgttggtaatccatatttt
agggttcctgcaggtggtggcaataagcaggatattcctaaggtttctgc
ataccaatatagagtatttagggtgcagttacctgacccaaataaatttg
gtttacctgatactagtatttataatcctgaaacacaacgtttagtgtgg
gcctgtgctggagtggaaattggccgtggtcagcctttaggtgttggcct
tagtgggcatccattttataataaattagatgacactgaaagttcccatg
ccgccacgtctaatgtttctgaggacgttagggacaatgtgtctgtagat
tataagcagacacagttatgtattttgggctgtgcccctgctattgggga
acactgggctaaaggcactgcttgtaaatcgcgtcctttatcacagggcg
attgcccccctttagaacttaaaaacacagttttggaagatggtgatatg
gtagatactggatatggtgccatggactttagtacattgcaagatactaa
atgtgaggtaccattggatatttgtcagtctatttgtaaatatcctgatt
atttacaaatgtctgcagatccttatggggattccatgtttttttgctta
cggcgtgagcagctttttgctaggcatttttggaatagagcaggtactat
gggtgacactgtgcctcaatccttatatattaaaggcacaggtatgcgtg
cttcacctggcagctgtgtgtattctccctctccaagtggctctattgtt
acctctgactcccagttgtttaataaaccatattggttacataaggcaca
gggtcataacaatggtgtttgctggcataatcaattatttgttactgtgg
tagataccactcgcagtaccaatttaacaatatgtgcttctacacagtct
cctgtacctgggcaatatgatgctaccaaatttaagcagtatagcagaca
tgttgaggaatatgatttgcagtttatttttcagttgtgtactattactt
taactgcagatgttatgtcctatattcatagtatgaatagcagtatttta
gaggattggaactttggtgttccccccccgccaactactagtttggtgga
tacatatcgttttgtacaatctgttgctattacctgtcaaaaggatgctg
caccggctgaaaataaggatccctatgataagttaaagttttggaatgtg
gatttaaaggaaagttttctttagacttagatcaatatccccttggacgt
aaatttttggttcaggctgga

The protein sequence of the HPV18 L1 used as basis is:

```
    1 MALWRPSDNT VYLPPPSVAR VVNTDDYVTR TSIFYHAGSS RLLTVGNPYF
  51 RVPAGGGNKQ DIPKVSAYQY RVFRVQLPDP NKFGLPDTSI YNPETQRLVW
101 ACAGVEIGRG QPLGVGLSGH PFY~KLDDTE SSHAATSNVS EDVRDNVSVD
151 YKQTQLCILG CAPAIGEHWA KGTACKSRPL SQGDCPPLEL KNTVLEDGDM
201 VDTGYGAMDF STLQDTKCEV PLDICQSICK YPDYLQMSAD PYGDSMFFCL
251 RREQLFARHF WNRAGTMGDT VPQSLYIKGT GMRASPGSCV YSPSPSGSIV
301 TSDSQLFNKP YWLHKAQGHN NGVCWHNQLF VTVVDTTRST NLTICASTQS
351 PVPGQYDATK FKQYSRHVEE YDLQFIFQLC TITLTADVMS YIHSMNSSIL
401 EDWNFGVPPP PTTSLVDTYR FVQSVAITCQ KDAAPAENKD PYDKLKFWNV
451 DLKEKFSLDL DQYPLGRKFL VQAGLRRKPT IGPRKRSAPS ATTSSKPAKR 500
```

In this connection, L1_{ΔC*} means an L1 protein with amino acids 1-474 of HPV18L1. L1_{ΔCDI} means an L1 protein with amino acids 1-472 and the additional HPV-foreign amino acids DI (Asp, Ile). The two amino acids AG underlined in the sequence above are thus replaced by DI in the L1_{ΔCDI} constructs.

The HPV18 E7 gene corresponds to the published DNA sequence and is for E7₁₋₆₄:

atgcatggacctaaggcaacattgcaagacattgtattgcatttagagcc
ccaaaatgaaattccggttgaccttctatgtcacgagcaattaagcgact
cagaggaagaaaacgatgaaatagatggagttaatcatcaacatttacca
gcccgacgagccgaaccacaacgtcacacaatgttgtaa

The protein sequence of the HPV18 E7 used as basis is:

1 MHGPKATLQD IVLHLEPQNE IPVDLLCHEQ LSDSEEENDE

IDGVNHQHLP

51 ARRAEPQRHT MLCMCCKCEA RIKLVVESSA DDLRAFQQLF

LNTLSFVCPW

101 CASQQ

Another method for obtaining the desired nucleic acids is to isolate the papillomavirus-specific genes directly from warts or tumors by means of PCR. Suitable primers for the E6 and E7 genes of HPV16 and HPV18 are disclosed for example in WO 93/21958. Further references for the desired nucleic acids are for example Kirnbaum, R. et al. (1994), supra and the clones deposited in the EMBL database which have already been mentioned above.

In a further preferred embodiment, the expression vector is constructed in such a way that the expressed fusion protein is extended by no further amino acids caused by the vector. This is achieved for example by deleting unwanted nucleotides which code for additional amino acids in a PCR reaction using suitable primer oligonucleotides (Ho et al. (1989) Gene, 77, 51-59). A fusion protein which is free of additional amino acids and is thus free of possibly additional foreign epitopes which may cause immunological side reactions is obtained in this way.

After expression of the described fusion protein, further purification or renaturation thereof is preferred. Examples of chromatographic purification methods are to be found in Hjorth, R. & Moreno-Lopez, L. (1982) J. Virol. Meth., 5, 151; Nakai, Y. et al. (1987), J. Gen. Virol., 68, 1891; Hofmann, K. J. et al. (1995) Virology, 209, 506; Rose, R. C. et al. (1993) J. Virol., 67, 1936, Sasagawa, T. et al. (1995) Virology, 206, 126 or WO 95/31532.

The medicament can generally be administered orally, parenterally, such as, for example, subcutaneously, intramuscularly or via the mucosa, in liquid or suspended form, in the form of an elixir or as capsules, preferably as solution for injection or infusion. It is possible to dispense with an adjuvant in the formulations of the invention, which is particularly advantageous.

A further aspect of the present invention therefore relates to the use of the formulation of the invention as solution for injection or infusion.

Solutions for injection are generally used when only relatively small amounts of a solution or suspension, for example about 1 to about 20 ml, are to be administered to the body. Solutions for infusion are generally used when a larger amount of a solution or suspension, for example one or more liters, are to be administered. Since, in contrast to the solution for infusion, only a few milliliters are administered in the case of solutions for injection, small differences from the pH and from the osmotic pressure of blood or of tissue fluid are not noticeable or are negligible in relation to the sensation of pain on injection. Dilution of the formulation of the invention before use is therefore generally unnecessary. By contrast, on administration of larger amounts, the formulation of the invention should be diluted shortly before administration so that an at least approximately isotonic solution is obtained. An example of an isotonic solution is 0.9% strength sodium chloride solution. On infusion, the dilution can take place for example with sterile water during the administration for example via a so-called bypass.

The figures and the following examples are intended to explain the invention in detail without restricting it.

### DESCRIPTION OF THE FIGURES

FIG. 1 shows a diagrammatic representation of the cloning of the HPV18 L1_{ΔC*}E7₁₋ₓ constructs, where X=55, 57, 62 or 64.

FIG. 2 shows a diagrammatic representation of the cloning of the HPV18 L1_{ΔC*}E7_{y-z} constructs, where Y=2 or 3, and Z=57, 60, 62 or 64.

FIG. 3A shows a graphical representation of absorption at 280 or 260 nm plotted against the time in minutes on an HPLC chromatogram of the HPV18 L1_{ΔC*}E7₂₋₆₂ fusion protein.

FIG. 3B shows the photograph of a silver staining of an SDS-PAGE analysis of the corresponding fractions of the chromatogram shown above it. The arrow indicates the main band of the HPV18 L1_{ΔC*}E7₂₋₆₂ fusion protein.

FIG. 4 shows the graphical analysis of two FACScan experiments after restimulation of specific murine T cells with LKK cells which present different peptides. The name of the respective peptide is indicated on the X axis; LKK cells without peptide were incubated merely with buffer and served as negative control. The proportion of CD8-positive T cells classified on the basis of IFNγ expression in the FACScan experiment as reactive is plotted on the Y axis.

FIG. 5 shows the graphical analysis of two FACScan experiments after restimulation of specific murine T cells with LKK cells which present different peptides. The name of the respective peptide is indicated on the X axis; LKK cells without peptide were incubated merely with buffer and served as negative control. The proportion of CD8-positive T cells classified on the basis of IFNγ expression in the FACScan experiment as reactive is plotted on the Y axis.

FIG. 6 shows the graphical analysis of a FACScan experiment after restimulation of specific human T cells with donor-identical BLCL which present different HPV18 peptide pools. The name of the respective peptide pool is indicated on the X axis; “without” stands for BLCL incubated only with buffer (negative control), L1 and E7 stand for BLCL incubated with HPV18 L1 and HPV18 E7 peptide pool, respectively (positive controls). The proportion of CD4-positive T cells classified on the basis of IFNγ expression in the FACScan experiment as reactive is plotted on the Y axis.

FIG. 7 shows the graphical analysis of a FACScan experiment after restimulation of specific human T cells with donor-identical BLCL which present the Q9 peptide. The name of the respective peptide is indicated on the X axis; BLCL stands for BLCL incubated only with buffer (negative control). The proportion of CD8-positive T cells classified on the basis of IFNγ expression in the FACScan experiment as reactive is plotted on the Y axis.

FIG. 8 shows the graphical analysis of the absorption in mAU at 280 and 270 nm plotted against the time in minutes on an HPLC chromatogram of the HPV18 L1_{ΔC*}E7₁₋₅₅ fusion protein.

FIG. 9 shows the graphical analysis of the absorption in mAU at 280 and 260 nm plotted against the time in minutes on an HPLC chromatogram of the HPV18 L1_{ΔC*}E7₁₋₆₄ fusion protein.

FIG. 10 shows the graphical analysis of a FACScan experiment after restimulation of specific murine T cells with JAWS II cells which have been loaded with CVLPs (HPV18L1_{ΔCDI}E7_{2-6DI} or HPV18L1_{ΔC}E7₁₋₆₄ in each case 10 or 40 μg) or with peptides (Q43, 44 or 45) (see X axis). JAWS II cells were incubated only with buffer (“without”) as negative control. The proportion of CD8-positive T cells classified on the basis of IFNγ expression in the FACScan experiment as reactive is plotted on the Y axis.

FIG. 11 shows the graphical analysis of a FACScan experiment after restimulation of specific murine T cells with JAWS II cells which have been loaded with CVLPs (HPV18L1_{ΔC}E7₁₋₆₄), native or boiled (“denatured”) or with the E7 peptide pool (see X axis). JAWS II cells were incubated only with buffer (“without”) as negative control. The proportion of CD8-positive T cells classified on the basis of IFNγ expression in the FACScan experiment as reactive is plotted on the Y axis.

### EXAMPLES

1. Production of Chimeric Genes Coding for HPV18L1E7 Fusion Proteins

Two primers complementary to HPV18L1 ORF were constructed to produce HPV18L1_{ΔCDI}. The first primer has the sequence

5′-ACC AGA CTC GAG ATG GCT TTG TGG CGG CCT AGT GAC-3′

and the second primer

5′-ATA GCC AAG CTT AAT GAT ATC CTG AAC CAA AAA TTT ACG TCC-3′

The first primer encodes 5′ an XhoI restriction enzyme cleavage site. The second primer encodes 5′ an EcoRV restriction enzyme cleavage site. The EcoRV site is followed by a TAA translation stop codon in order to delete the last 35 amino acids of the HPV18L1 ORF. The PCR product was cleaved with XhoI/EcoRV and ligated into the likewise XhoI/EcoRV-cleaved pBluescript® vector. The resulting construct HPV18L1_{ΔC} pKS was used to clone the ORF of HPV18E7₁_{1-53DI} and HPV18E7_{1-60DE} into the EcoRV site.

Primers with a 5′EcoRV restriction enzyme cleavage site were used to clone the HPV18 E7 fragments. The following pairs of primers were used:

| | |
|---|---|
| 5′-ACC AGA CTC GAG ATG GCT TTG TGG CGG CCT AGT GAC-3′ | (5′end of the E7 gene) and |
| 5′-GGC CAT GAT ATC TCG TCG GGC TGG TAA ATG TTG ATG-3′ | (3′end of the E7_{1-53DI} fragment) or |
| 5′-GGC CAT GAT ATC TGT GTG ACG TTG TGG TTC GGC TC-3′ | (3′end of the E7_{1-60DI} fragment). |

The PCR products were cleaved with EcoRV and inserted into the EcoRV site of the modified L1 gene.

The EcoRV sites in the HPV18 L1_{ΔC}E7₁₋ₓ were eliminated by using the fact that the first two amino acids of the E7 protein (Met-His) correspond at the DNA level to the recognition sequence of the restriction enzyme NsiI (ATG CAT).

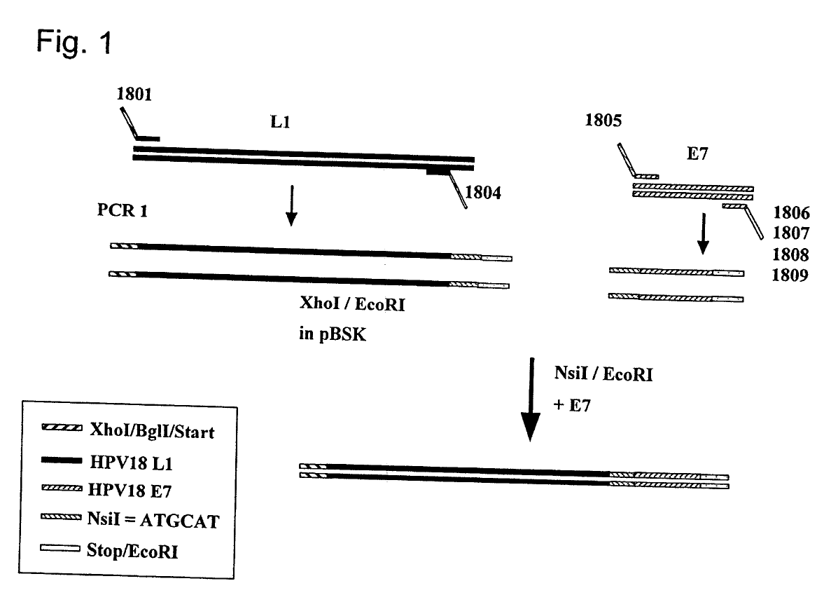

Firstly, an HPV18 L1_{ΔC}E7₁₋₂ fragment was produced by a PCR reaction. The 5′ primer 1801 encoded an XhoI cleavage site followed by a BglII cleavage site, the start codon and the first nucleotides of the HPV18 L1 gene. The 3′ primer 1804 coded for the last nucleotides of the HPV18 LLAC gene followed by an NsiI cleavage site which represents the first six nucleotides of the HPV18 E7 gene, and by an EcoRI cleavage site. The resulting PCR fragment was cut with the XhoI/EcoRI enzymes and cloned into the pBluescript® (Stratagene) vector. The resulting vector was called pBSK-18L1_{ΔC}E7₁₋₂ (see FIG. 1).

1801: 5′-CGC CGC CTC GAG AGA TCT ATG GCT TTG TGG CGG CCT-3′
1804: 5′-CCG GAA TTC CCA CCA ATG CAT TCC AGC CTG AAC CAA AAA TTT-3′

The fragments for HPV18 E7 1-55, 1-57, 1-62 and 1-64 were likewise produced by PCR reactions. In these cases, the same 5′ primer 1805 which coded for the first 24 nucleotides of the HPV18 E7 gene, the first six of these representing the NsiI cleavage site, was used for all the fragments. The 3′ primers 1806, 1807, 1808 and 1809 coded for the last 21 nucleotides of the respective HPV18 E7 fragments followed by a stop codon and an EcoRI cleavage site. The resulting PCR fragments were cleaved with NsiI/EcoRI and ligated into the NsiI/EcoRI-cut vector pBSK-18L1_{ΔC}E7₁₋₂ to result in the fusion genes HPV18 L1_{ΔC}E7₁₋₅₅, HPV18 L1_{ΔC}E7₁₋₅₇, HPV18 L1_{ΔC}E7₁₋₆₂, HPV18 L1_{ΔC}E7₁₋₆₄ in the pBluescript® vector (see FIG. 1).

1805:
5′-CGC GGA TCC ATG CAT GGA CCT AAG GCA ACA TTG-3′
1806:
5′-CCG GAA TTC TTA TTC GGC TCG TCG GGC TGG TAA-3′
1807:
5′-CCG GAA TTC TTA TTG TGG TTC GGC TCG TCG GGC-3′
1808:
5′-CCG GAA TTC TTA CAA CAT TGT GTG ACG TTG TGG-3′
1809:
5′-CCG GAA TTC TTA CAT ACA CAA CAT TGT GTG ACG-3′

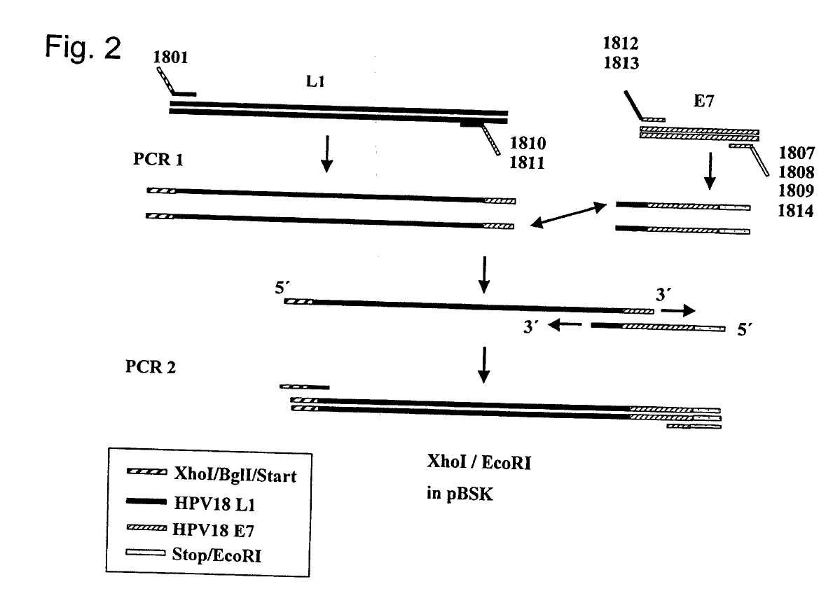

The NsiI cleavage site could not be used to produce fusion genes whose E7 portion does not start until amino acid 2 or 3. These constructs were produced by carrying out two PCR reactions. The product of the first reaction was the L1_{Δ*} gene to which the start of the E7 gene (starting at amino acid 2 or 3) was fused. The product of the second reaction is the E7 gene (starting at amino acid 2 or 3), in front of which the end of the L1_{ΔC*} gene was fused. The resulting DNA fragments thus overlapped at the position of the L1/E7 boundary (“Four Primer PCR”, Ho, S. N. et al (1989) Gene 77, 51). However, the primers contained no restriction enzyme cleavage sites. Fragment 1 of the L1_{ΔC*}E7₂₋ₓ fusion genes was produced using the primer combination 1801/1810 and fragment 2 using the primer combinations 1812/1807 (L1_{ΔC*}E7₂₋₅₇) and 1812/1814 (L1_{ΔC*}E7₂₋₆₀) and 1812/1808 (L1_{ΔC*}E7₂₋₆₂). The L1_{ΔC*}E7₃₋₆₄ fusion gene was produced by the same method. In this construct, fragment 1 was produced using the primer combination 1801/1811 and fragment 2 using the primer combination 1813/1809 (see FIG. 2).

1810: 5′-TTG CAA TGT TGC CTT AGG TCC ATG TCC AGC CTG AAC CAA AAA TTT-3′
1811: 5′-GTC TTG CAA TGT TGC CTT AGG TCC TCC AGC CTG AAC CAA AAA TTT-3′
1812: 5′-AAA TTT TTG GTT CAG GCT GGA CAT GGA CCT AAG GCA ACA TTG CAA-3′
1813: 5′-AAA TTT TTG GTT CAG GCT GGA GGA CCT AAG GCA ACA TTG CAA GAC-3′

One tenth of the respective purified products was mixed and used as template in the PCR reaction exclusively with the primer combinations 1801/1807 (L1_{ΔC*}E7₂₋₅₇) and 1801/1814 (L1_{ΔC*}E7₂₋₆₀), 1801/1808 (L1_{ΔC*}E7₂₋₆₂) and 1801/1809 (L1_{ΔC*}E7₃₋₆₄). The resulting products was cleaved with XhoI (upstream from the start codon) and EcoRI (downstream from the stop codon) and ligated into the XhoI/EcoRI-cut pBluescript® vector.

The resulting HPV18 L1_{ΔC*}E7_{x-y} constructs therefore differ from the clones HPV18 L1_{ΔCDI} E7_{1-53DI} and HPV18 L1_{ΔCDI} E7_{1-60DI} through loss of the two internal EcoRV restriction enzyme cleavage sites and of the corresponding non-HPV amino acids Asp and Ile between the L1 ORF and E7 and downstream from E7. The first EcoRV site was replaced by the original L1 amino acids at this position (AlaGly). The second EcoRV site was replaced by a translation stop signal.

The clones were analyzed by DNA sequencing. The HPV18 L1_{ΔC*}E7_{x-y} fusion genes were then cut out of the pBluescript vector by BglII/EcoRI restriction digestion and ligated into the BglII/EcoRI-cleaved baculovirus transfer vector pVL1392 in order to produce recombinant baculoviruses.

2. Production of Recombinant Baculoviruses Spodoptera frugiperda (Sf9) cells were grown as monolayer or in suspension culture in TNM-FH insect medium (Life Technologies, Karlsruhe) with 10% fetal calf serum. Recombinant baculoviruses were produced by cotransfection of 5 μg of the recombinant plasmids and 1 μg of linearized Baculo-Gold® DNA (Pharmingen, San Diego, Calif.) in Sf9 cells. Recombinant viruses were purified by endpoint dilution and/or plaque isolation. In order to test the expression, 10⁶ Sf9 cells were infected with recombinant baculovirus and an m.o.i. (“multiplicity of infection”) of 0.5 and 1 for 48 h. After the incubation, the medium was removed and the cells were washed with PBS (140 mM NaCl, 2.7 mM KCl, 8.1 mM Na₂HPO₄, 1.5 mM KH₂PO4, pH 7.2). The cells were then investigated by a FACS measurement or lyzed in SDS sample buffer and assayed by SDS gel chromatography and immunoblot assay.

3. Purification of Virus-like Particles

CVLPs were produced by culturing Sf9 or SF+ cells in the serum-free medium InsectXPress (Biowhittaker, Verviers, Belgium) or Sf 900II (Life Technologies, Karlsruhe) at 27° C. to a density of 1.5-2×10⁶ cells per ml. A 200 ml culture was infected with an m.o.i. of 1 to 2 with recombinant baculoviruses for 48 h. The cells were then pelleted and frozen at −80° C. Freeze-thaw lysis took place by adding 4 vol. of extraction buffer (200 mM NaCl, 50 mM Tris, pH 8.5). The homogenate was clarified by centrifugation at 10 000 rpm in a Sorvall SS34 rotor. The L1E7 protein was purified from the clarified crude extract for the immunological assays by ammonium sulfate precipitation at 35-40% saturation and subsequent anion exchange chromatography on Fractogel® TMAE (Merck, Darmstadt), with the CVLPs being eluted in the linear salt gradient at 300-400 mM NaCl. The protein content of the purified fractions was determined by the Bradford method using bovine serum albumin as standard.

4. Capsid Detection in Purified HPV18 L17 Fractions

It was possible even beforehand to show for HPV16 L1_{ΔC}E7₁₋₅₅ CVLPs by combination with sucrose density gradient centrifugation and transmission electron microscopy that after a size exchange chromatography on a TSK gel G6000PX_{XL} column (Tosoh Haas, Stuttgart) in an Agilent 1100 HPLC system a retention time of about 10 +/−0.5 min (flow rate 0.8 ml/min) is specific for corresponding capsids.

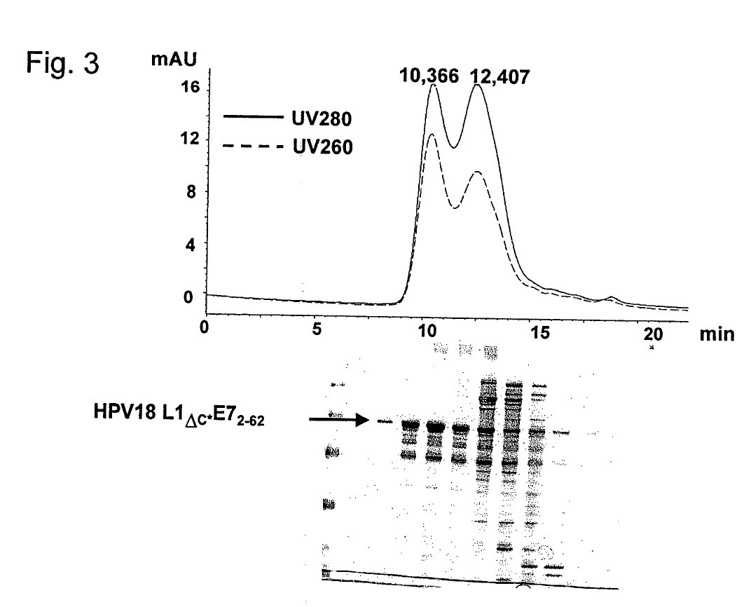

It now emerged on analysis of the HPV18 L1_{ΔC*}E7₂₋₆₂ fusion protein that the proteins purified as in example 3 eluted with a retention time of 10.366 min after a size exchange chromatography under identical conditions as for the HPV16 capsids (see FIG. 3A). It was possible to confirm the presence of the HPV18 L1E7 fusion proteins in the fractions by SDS-PAGE analysis (see FIG. 3B) and immunoblot (data not shown). A second peak at 12.407 min still contains part of the construct and a number of impurities. FIG. 3 thus shows that the construct HPV18 L1_{ΔC*}E7₂₋₆₂ is present to a considerable extent in capsids under these conditions.

Particle formation was likewise detectable for the following constructs:

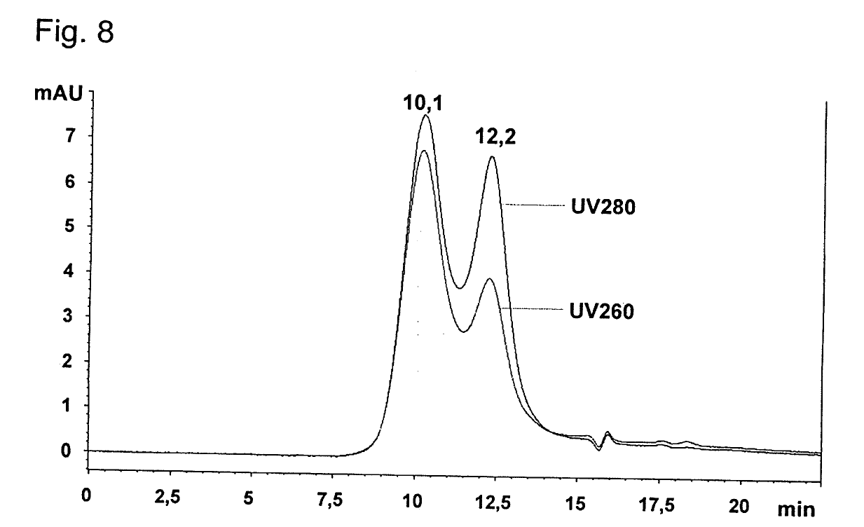

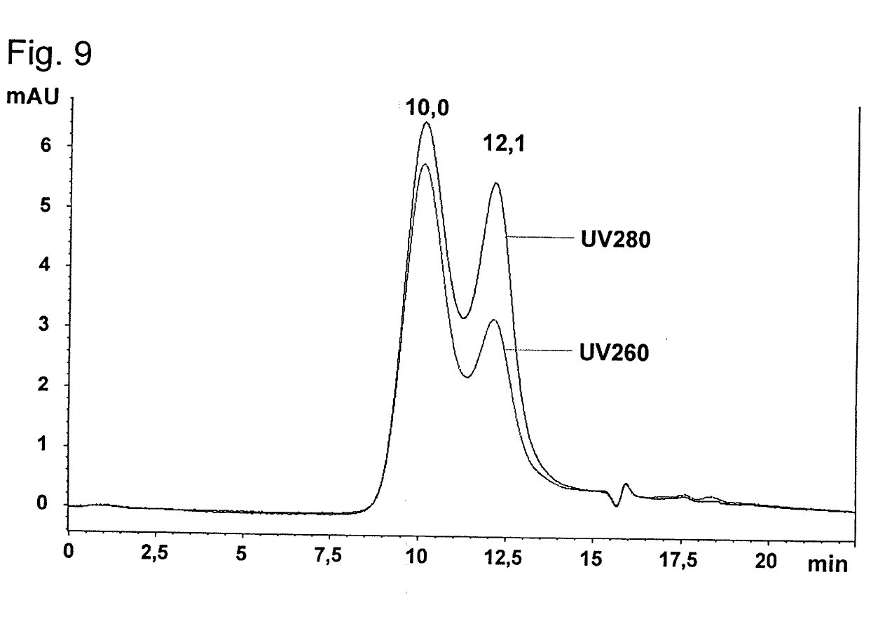

L1_{ΔC*}E7₁₋₅₅ (see FIG. 8), L1_{ΔC*}E7₁₋₅₇, L1_{ΔC*}E7₁₋₆₂, L1_{ΔC*}E7₁₋₆₄ (see FIG. 9), L1_{ΔC*}E7₃₋₆₄, L1_{ΔCDI}E7_{1-53DI}, and L1_{ΔCDI}E7_{1-60DI}.

It is surprising in this connection that the construct HPV18 L1_{ΔC*}E7₁₋₆₄ forms capsids just as efficiently as the considerably shorter construct HPV18 L1_{ΔC*}E7₁₋₅₅ (see FIG. 8 and 9), because on the basis of the known data for HPV16 capsid formation (see Müller M. et al, 1997, supra) no particle formation would be expected for such long E7 portions with C-terminal cysteines.

5. Immunogenicity of HPV18 L1_{ΔCDI}E7_{1-53DI} CVLPs and HPV18 L1_{ΔCDI}E7_{1-60DI} CVLPs in Mice

Several C₃H mice were immunized twice with 20 μg each time of a 1:1 mixture of HPV18 L1_{ΔCDI}E7_{1-53DI} CVLPs and HPV18 L1_{ΔCDI}E7_{1-60DI} CVLPs and with buffer as control. After 2 weeks, the spleen cells were obtained by standard methods (murine T cells).

20mer peptides which comprise the sequence of the L1 and of the E7 protein of HPV18 and which overlap by 9 amino acids in each case were then synthesized. The peptides were numbered from 1 to 52 consecutively. Their name and their sequence are summarized in the following table.

TABLE 1

Synthetic overlapping 20mer peptides of HPV18
L1 and E7

| | |
|---|---|
| Peptide Sequence | Relative |
| No. | position |

HPV18 L1 Peptides

| | | |
|---|---|---|
| Q1 | MALWRPSDNTVYLPPPSVAR | 1-20 |
| Q2 | YLPPPSVARVVNTDDYVTRT | 12-31 |
| Q3 | NTDDYVTRTSIFYHAGSSRL | 23-42 |
| Q4 | FYHAGSSRLLTVGNPYFRVP | 34-53 |
| Q5 | VGNPYFRVPAGGGNKQDIPK | 45-64 |
| Q6 | GGNKQDIPKVSAYQYRVFRV | 56-75 |
| Q7 | AYQYRVFRVQLPDPNKFGLP | 67-86 |
| Q8 | PDPNKFGLPDTSIYNPETQR | 78-97 |
| Q9 | SIYNPETQRLVWACAGVEIG | 89-108 |
| Q10 | WACAGVEIGRGQPLGVGLSG | 100-119 |
| Q11 | QPLGVGLSGHPFYNKLDDTE | 111-130 |
| Q12 | FYNKLDDTESSHAATSNVSE | 122-141 |
| Q13 | HAATSNVSEDVRDNVSVDYK | 133-152 |
| Q14 | RDNVSVDYKQTQLCILGCAP | 144-163 |
| Q15 | QLCILGCAPAIGEHWAKGTA | 155-174 |
| Q16 | GEHWAKGTACKSRPLSQGDC | 166-185 |
| Q17 | SRPLSQGDCPPLELKNTVLE | 177-196 |
| Q18 | LELKNTVLEDGDMVDTGYGA | 188-207 |
| Q19 | DMVDTGYGAMDFSTLQDTKC | 199-218 |
| Q20 | FSTLQDTKCEVPLDICQSIC | 210-229 |
| Q21 | PLDICQSICKYPDYLQMSAD | 221-240 |
| Q22 | PDYLQMSADPYGDSMFFCLR | 232-251 |
| Q23 | GDSMFFCLRREQLFARHFWN | 243-262 |
| Q24 | QLFARHFWNRAGTMGDTVPQ | 254-273 |
| Q25 | GTMGDTVPQSLYIKGTGMRA | 265-284 |
| Q26 | YIKGTGMRASPGSCVYSPSP | 276-295 |
| Q27 | GSCVYSPSPSGSIVTSDSQL | 287-306 |
| Q28 | SIVTSDSQLFNKPYWLHKAQ | 298-317 |
| Q29 | KPYWLHKAQGHNNGVCWHNQ | 309-328 |
| Q30 | NNGVCWHNQLFVTVVDTTRS | 320-339 |
| Q31 | VTVVDTTRSTNLTICASTQS | 331-350 |
| Q32 | LTICASTQSPVPGQYDATKF | 342-361 |
| Q33 | PGQYDATKFKQYSRHVEEYD | 353-372 |
| Q34 | YSRHVEEYDLQFIFQLCTIT | 364-383 |
| Q35 | FIFQLCTITLTADVMSYIHS | 375-394 |
| Q36 | ADVMSYIHSMNSSILEDWNF | 386-405 |
| Q37 | SSILEDWNFGVPPPPTTSLV | 397-416 |
| Q38 | PPPPTTSLVDTYRFVQSVAI | 408-427 |
| Q39 | YRFVQSVAITCQKDAAPAEN | 419-438 |
| Q40 | QKDAAPAENKDPYDKLKFWN | 430-449 |
| Q41 | PYDKLKFWNVDLKEKFSLDL | 441-460 |
| Q42 | LKEKFSLDLDQYPLGRKFLV | 452-471 |
| Q43 | YPLGRKFLVQAGMHGPKATL | 463-474 |
| | | E7 and |
| | | 1-8 |

HPV18 E7 Peptides

| | | |
|---|---|---|
| Q44 | MHGPKATLQDIVLHLEPQNE | 1-20 |
| Q45 | VLHLEPQNEIPVDLLCHEQL | 12-31 |
| Q46 | VDLLCHEQLSDSEEENDEID | 23-42 |
| Q47 | SEEENDEIDGVNHQHLPARR | 34-53 |
| Q48 | NHQHLPARPAEPQRHTMLCM | 45-64 |
| Q49 | PQRHTMLCMCCKCEARIKLV | 56-75 |
| Q50 | KCEARIKLVVESSADDLRAF | 67-86 |
| Q51 | SSADDLRAFQQLFLNTLSFV | 78-97 |
| Q52 | LFLNTLSFVCPWCASQQ | 89-104 |

HPV18 L1 peptide pools mean the mixture of the peptides Q1 to Q43, and HPV18 E7 peptide pools mean the mixture of the peptides Q44 to Q52.

The murine T cells (4×10⁵) of HPV18 L1_{ΔCDI}E7_{1-53DI} CVLP-and HPV18 L1_{ΔCDI}E7_{1-60DI} CVLP-immunized C₃H mice were stimulated for 5 weeks with HPV18 L1 or E7 peptide pools at 37° C. with weekly addition of 1 μg/ml of each individual peptide and 10⁵ antigen-presenting cells (irradiated splenocytes, obtained by standard methods from the spleen of unimmunized mice) and harvested, the cells were then restimulated in 100 μl of medium at 37° C. with 1 μg/ml of the peptides indicated on the X axis of FIG. 3, and 10⁵ antigen-presenting cells (LKK, from ATCC, number CCL-1) in the presence of 10 IU/ml IL2 (Becton Dickinson, Hamburg). Cells incubated only with buffer served as negative control.

After one hour, 1 μl of monensin (300 μM, Sigma, Deisenhofen) were added. The cells were incubated at 37° C. for a further 5 hours. The cells were then fixed and permeabilized, stained with α-mouse CD8/PE (monoclonal rat antibodies against the extracellular part of the murine CD8 coupled to the fluorescent marker phycoerythrin, Pharmingen, Heidelberg), with α-mouse CD4/Cychrome (monoclonal rat antibody directed against the extracellular part of the murine CD4, coupled to Cychrome, Pharmingen, Heidelberg) and with α-mouse IFNγ/FITC (monoclonal rat antibody against murine interferon γ coupled to FITC, Caltag, Hamburg). The cells were investigated for their labeling in a FACScan calibur (fluorescence activated cell sorter, Becton Dickinson, Hamburg), and the measured results were analyzed with the aid of Cellquest software (Becton Dickinson, Hamburg).

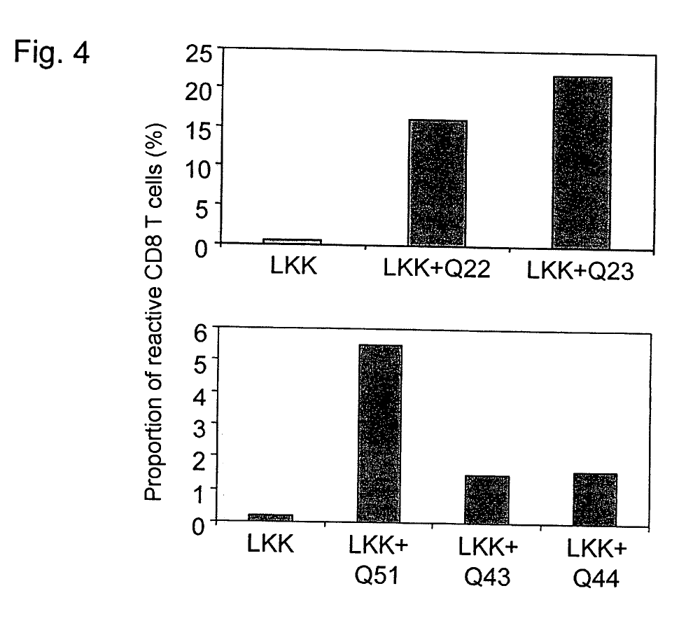

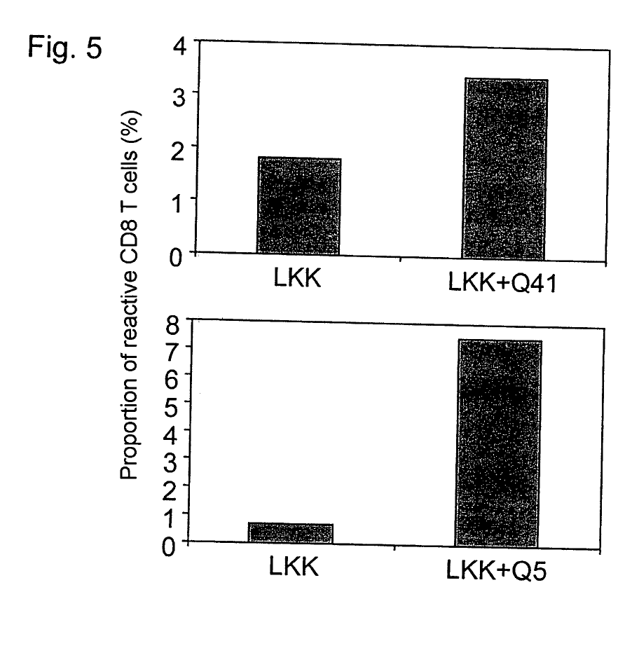

Result: FIG. 4 shows for the peptides Q22, Q23, Q51, Q43 and Q44, and FIG. 5 for the peptides Q41 and Q5, that the LKK cells incubated with the peptides brought about restimulation of peptide-stimulated murine CD8-positive T cells. This means that the mice immunized with the HPV18 L1_{ΔCDI}E7_{1-53DI} CVLPs and HPV18 L1_{ΔCDI}E7_{1-60DI} CVLPs have produced cytotoxic T cells which are directed both against peptides from L1 (Q5, Q22, Q23, Q41, Q43) and peptides from E7 (Q44, Q51). This therefore demonstrates a cellular immune response mediated by cytotoxic T cells after immunization with the HPV18 constructs in mice.

6. Generation of HPV18-specific Human T-helper Cells

Human T cells (4×10⁵) from a non-HLA-typed blood donor were stimulated for 1 week with HPV18 L1_{ΔCDI}E7_{1-53DI} CVLPs and HPV18 L1_{ΔCDI}E7_{1-60DI} CVLPs, 800 U/ml of human GM-CSF (Leukomax, Novartis Pharma GmbH, Nürnberg) and 500 U/ml of human IL4 (Becton Dickinson, Hamburg) and for a further 5 weeks with HPV18 L1_{ΔCDI}E7_{1-53DI} CVLPs and HPV18 L1_{ΔCDI}E7_{1-60DI} CVLPs at 37° C. with weekly addition of 1 μg/ml of the CVLPs mixture (ratio 1:1 of the two constructs) and 10⁵ antigen-presenting cells (irradiated PBMC, peripheral blood mononuclear cells, isolated by the method of Rudolf M. P. et al (1990), Biol. Chem. 380, 335-40)and harvested.

The 20mer peptides Q1 to 52 from example 5 were combined in accordance with the matrix

| HPV18 pools | A | B | C | D | E | F | G | H |
|---|---|---|---|---|---|---|---|---|
| 1 | Q1 | Q2 | Q3 | Q4 | Q5 | Q6 | Q7 | Q8 |
| 2 | Q9 | Q10 | Q11 | Q12 | Q13 | Q14 | Q15 | Q16 |
| 3 | Q17 | Q18 | Q19 | Q20 | Q21 | Q22 | Q23 | Q24 |
| 4 | Q25 | Q26 | Q27 | Q28 | Q29 | Q30 | Q31 | Q32 |
| 5 | Q33 | Q34 | Q35 | Q36 | Q37 | Q38 | Q39 | Q40 |
| 6 | Q41 | Q42 | Q43 | Q44 | Q45 | Q46 | Q47 | Q48 |
| 7 | Q49 | Q50 | Q52 | Q52 | | | | |

into peptide pools A to H and 1 to 7.

The T cells were then restimulated in 100 μl of medium at 37° C. with the peptide pools and 10⁵ antigen-presenting cells (donor-identical BLCL, B-cell line transformed in each case with the aid of the Epstein-Barr virus and produced individually for each blood donor, obtained from Dr A. Kaufmann, Jena) in the presence of 10 IU/ml IL2 (Becton Dickinson). The amounts of the peptide pools employed for this were such that 1 μg/ml was added for each individual peptide. Cells incubated only with buffer served as negative control, and cells incubated with HPV18 L1 or HPV18 E7 peptide pools served as positive controls.

After one hour, 1 μl of monensin was added. The cells were incubated at 37° C. for a further 5 hours. The cells were then fixed and permeabilized, stained with a-human CD8/APC (monoclonal mouse antibody directed against the extracellular part of the human CD8, coupled to the fluorescent marker APC, Caltag, Hamburg), with α-human CD4/PerCP (monoclonal mouse antibody directed against the extracellular part of the human CD4, coupled to the fluorescent marker PercP, Becton Dickinson, Hamburg) and with α-human IFNγ/FITC (monoclonal mouse antibody directed against human interferon α, coupled to the fluorescent marker FITC, Caltag, Hamburg). The cells were investigated for their labeling in a FACScan calibur, and the measured results were analyzed with the aid of Cellquest software.

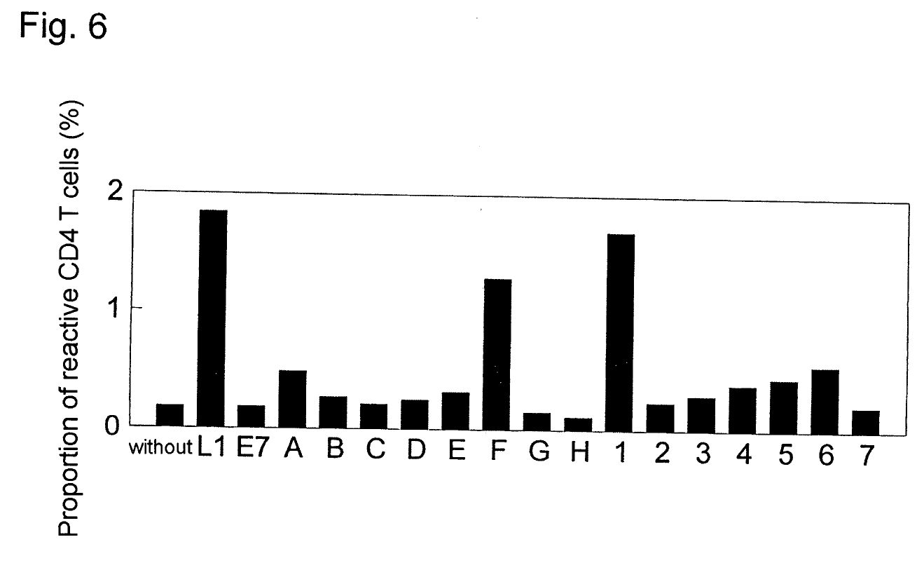

Result: FIG. 6 shows that, in particular, the BLCL incubated with the peptide pools L1, F and 1 brought about restimulation of human CD4-positive T cells which had been stimulated with HPV18 L1_{ΔCDI}E7_{1-53DI} CVLPs and HPV18 L1_{ΔCDI}E7_{1-60DI} CVLPs. In addition, peptide pools A and 6 showed restimulation distinctly exceeding the negative control. The BLCL incubated with the other peptide pools, and the negative control or the BLCL incubated with the E7 peptide pool by contrast showed a small proportion of reactive CD4-positive T cells. The peptide pools F and 1 jointly contain the peptide Q6, which is thus most likely responsible for the restimulation of the CVLP-stimulated T cells. This means that it was possible to produce HPV18 L1-specific T-helper cells, which—in this case—are directed against the L1 peptide Q6, by incubation of HPV18 L1_{ΔCDI}E7_{1-53DI} CVLPs, HPV18 L1_{ΔCDI}E7_{1-60DI} CVLPs and human T cells. HPV18 constructs of this type are able to induce a cellular immune response mediated by T-helper cells against L1 and E7 after immunization.

7. Generation of HPV18-specific Human Cytotoxic T Cells

In analogy to example 6, human T cells (4×10⁵) of an HLA A24-positive donor were stimulated for 3 weeks with the HPV18 L1 peptide pool at 37° C. with weekly addition of 1 μg/ml of each individual peptide and 10⁵ antigen-presenting cells (irradiated PBMC) and harvested.

The cells were then restimulated in 100 μl of medium at 37° C. with 10 μg/ml of the HPV18 L1 peptide Q9 and 10⁵ antigen-presenting cells (donor-identical BLCL) in the presence of 10 IU/ml IL2. Cells incubated only with buffer served as negative control.

After one hour, 1 μl of monensin (300 μM) were added. The cells were incubated at 37° C. for a further 5 hours. The cells were then fixed and permeabilized, stained with α-human CD8/APC, with α-human CD4/PerCP and with α-human IFNγ/FITC. The cells were investigated for their labeling in a FACScan calibur and the measured results were analyzed with the aid of Cellquest software.

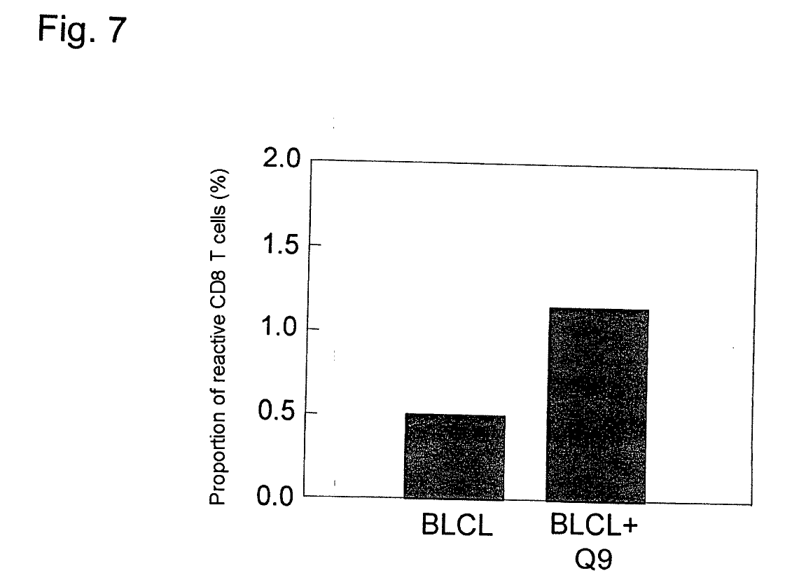

Result: FIG. 7 shows that the BLCL cells incubated with the peptide Q9 brought about restimulation of HPV18 L1 peptide pool-stimulated CD8-positive T cells. It was found by means of the algorithm for potential HLA A24-binding peptides (Parker, K C et al., (1994) J. Immunol. 152:163) carried out in the so-called peptide prediction program of Parker under http://www-bimas.dcrt.nih.gov/molbio/hla_bind/ that the peptide IYNPETQRL binds to MHC class I molecules of the HLA A24 haplotype. It can thus be assumed that the peptide IYNPETQRL is responsible for the restimulation of the T cells by the BLCL cells incubated with the Q9 peptide. This means that it was possible to produce HPV18 L1-specific cytotoxic T cells, which—in this case—are directed against the L1 peptide Q9, by incubation of HPV18 L1_{ΔCDI}E7_{1-53DI} CVLPs and HPV18 L1_{CDI}E7_{1-60DI} CVLPs and human T cells. HPV18 constructs of this type are able to induce a cellular immune response against L1 and E7 which is mediated by cytotoxic T cells after immunization.

8. Detection of a CTL Immune Response After Immunization of Black6 Mice with HPV18 Constructs

Several C57/BL/6 mice were immunized twice with 20 μg of HPV18 L1_{ΔCDI}E7_{2-62DI} CVLPs each time and with buffer as control. The removal of the spleens, the isolation of the splenocytes and the cultivation and restimulation therefor for the purposes of FACScan analysis were carried out as described in example 5, 6 and 7.

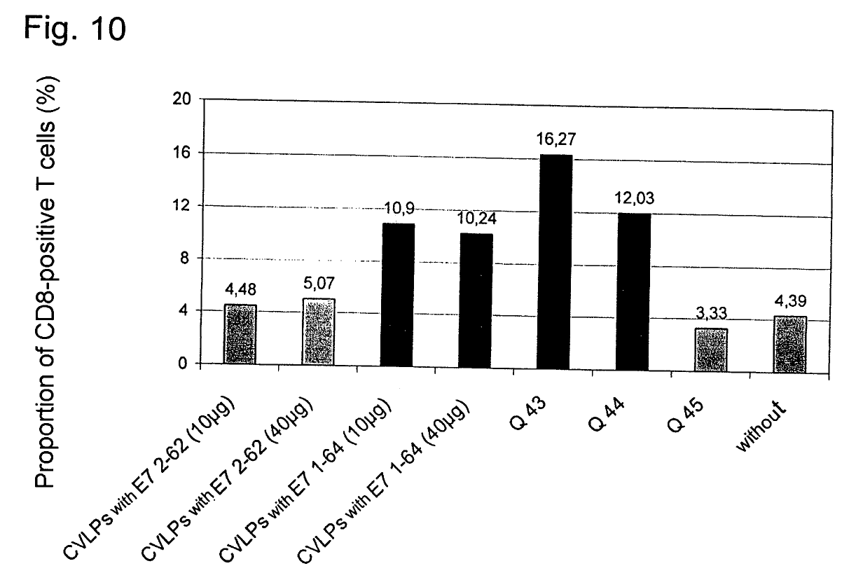

Thus, murine T cells (JAWS-II) were stimulated with weekly addition of HPV18 L1 and E7 peptide pools and the irradiated splenocytes obtained from the mice, and harvested. The cells were then restimulated with the CVLPs (HPV18 L1_{ΔCDI}E7_{2-62DI} or HPV18 L1_{ΔCDI}E7₁₋₆₄, in each case 10 or 40 μg) and peptides (Q43, Q44 or Q45) indicated on the X axis of FIG. 10 as described in example 5 at 37° C. overnight. Cells incubated only with buffer served as negative control.

Evaluation of the experiment took place as in example 5 via measurement of the CD8 and of the INF-γ.

Result: FIG. 10 shows that JAWS-II cells which were incubated with HPV18 L1_{ΔC}E7₁₋₆₄ CVLPs, in contrast to the HPV18 L1_{ΔCDI}E7_{2-62DI} CVLPs, brought about restimulation of peptide-stimulated murine CD8-positive T cells in the intracellular IFN-γ assay. A similar restimulation was observable for the peptides Q43 and Q44 but not for the peptide Q45. The peptides Q43 and Q44 jointly comprise the amino acids MHGPKATL which correspond to the N-terminal amino acids of E7. This experiment thus indicates that the amino acids MHGPKATL comprise an H2 b restricted cytotoxic T-cell epitope which is likewise recognized by the HPV18 L1_{ΔC}E7₁₋₆₄ CVLPS. This means that omission of the M (position 1 of E7) alone presumably leads to the HPV18 L1_{ΔCDI}E7_{2-62DI} CVLPs no longer being able to induce a cytotoxic T-cell response under the given experimental conditions. This is all the more surprising since the prediction by the so-called peptide prediction program of Parker et al. (1994), J. Immunol. 152:163, under http://www-bimas.dcrt.nih/ gov/Molbio/hla_bind/ predicts no murine T-cell epitope starting with this M.

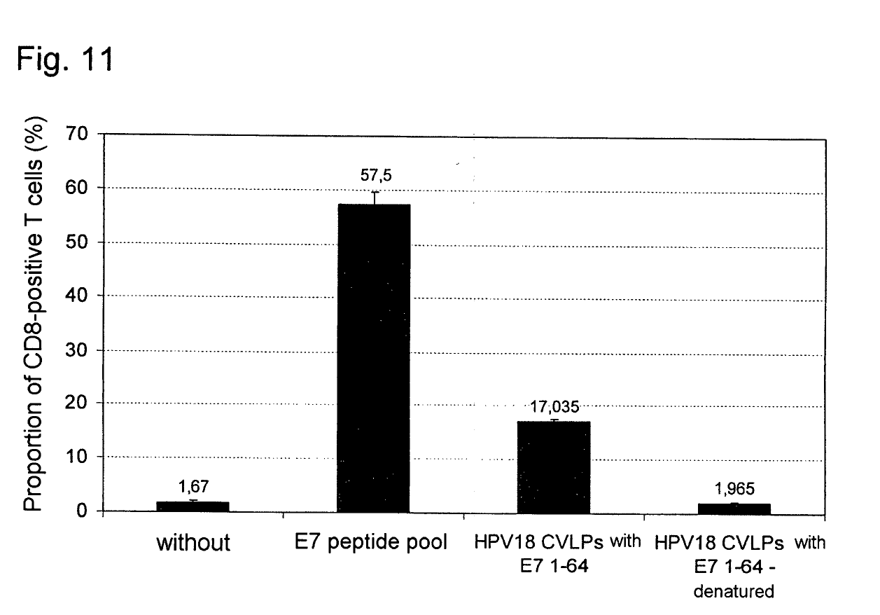

It was also possible to demonstrate that the measured T-cell activation was specific for the CVLPs since, under identical experimental conditions comparing between CVLPs of the type HPV18 L1_{ΔC}E7₁₋₆₄, the activation of the T cells was lost due to denaturation of the CVLPs by boiling (see FIG. 11).

1. A medicament for the prevention or treatment of human papillomavirus type 18 (HPV-18)-specific tumor comprising at least one fusion protein of at least one L protein and at least one E protein of one or more HPV-18 and, where appropriate, suitable additives and/or excipients, characterized in that the fusion protein is an L1ΔCE7_{x-y} fusion protein, where x is an integer from 1 to 3 inclusive and y is an integer from 61 to 64, in particular 62 to 64, especially 62 or 64.

2. The medicament as claimed in claim 1, characterized in that the fusion protein is a CVLP.

3. The medicament as claimed in claim 1 or 2, characterized in that the fusion protein comprises no foreign protein content or papillomavirus-nonspecific epitopes.

4. The medicament as claimed in claim 1 or 2, characterized in that the fusion protein comprises a foreign protein content or papillomavirus-nonspecific epitopes.

5. The medicament as claimed in any of claims 1 to 4, characterized in that the tumor is a laryngeal, cervical, penile, vulval or anal carcinoma.

6. The medicament as claimed in any of claims 1 to 4, characterized in that the medicament comprises no adjuvant.

7. The medicament as claimed in any of claims 1 to 6, characterized in that the additive or excipient is about 0.1 to about 3 M, preferably about 0.15 to about 1 M, in particular about 0.2M of a salt having a pH of about 7 to about 8, preferably of about 7.3 to 7.4, in particular of 7.4.

8. The medicament as claimed in claim 7, characterized in that the salt is an alkali metal or alkaline earth metal salt, preferably a halide or phosphate, in particular an alkali metal halide, especially NaCl and/or KC1.

9. The medicament as claimed in claim 7 or 8, characterized in that the pH is adjusted with a buffer, preferably with a phosphate buffer, Tris buffer, HEPES buffer or MOPS buffer.

10. The medicament as claimed in any of claims 1 to 9, characterized in that the L protein is a deleted L protein, preferably a deleted L1 and/or L2 protein.

11. The medicament as claimed in claim 10, characterized in that the L protein is a C-terminally deleted L protein, in particular a C-terminally deleted L1 protein.

12. The medicament as claimed in claim 10 or 11, characterized in that up to about 35 amino acids are deleted from the L protein, preferably about 15 to about 35 amino acids, in particular about 26-28 amino acids.

13. The medicament as claimed in any of claims 1 to 12, characterized in that the E protein is a deleted E protein, in particular a deleted E6 and/or E7 protein.

14. The medicament as claimed in claim 13, characterized in that the deleted E protein is a C-terminally deleted E protein, preferably a C-terminally deleted E7 protein.

15. The medicament as claimed in claim 13 or 14, characterized in that up to about 55 amino acids are deleted, preferably about 5 to about 55 amino acids, in particular about 40 to 51 amino acids, especially about 41 to 45 amino acids.

16. The medicament as claimed in claim 13, characterized in that the deleted E protein is an N-terminally deleted E protein, preferably an N-terminally deleted E7 protein.

17. The medicament as claimed in claim 12 or 15, characterized in that up to about 10 amino acids are deleted, preferably 1 to 2 amino acids.

18. The medicament as claimed in any of claims 1 to 17, characterized in that the fusion protein is in the form of a capsid and/or capsomere.

19. The medicament as claimed in any of claims 1 to 18 in the form of a solution for injection or infusion.

20. A method for producing a medicament as claimed in any of claims 1 to 19, characterized in that a cell comprising an expression vector which codes for said fusion protein is cultivated under suitable conditions, the expression product is isolated, and suitable additives and excipients are added where appropriate.

21. The method as claimed in claim 20, characterized in that the expression vector comprises no nucleic acid sequences which extend said fusion protein by further amino acids on expression.

22. The use of a fusion protein as set forth in any of claims 1 to 19 for producing a medicament on the one hand for the prevention of HPV-specific tumors, and on the other hand for the regression of already existing HPV-specific tumors.


SEQUENCE LISTING (72 sequences)

SEQ ID NO: 1 - DNA, 1421 bp, Human papillomavirus type 18
atggctttgtggcggcctagtgacaataccgtatatcttccacctccttctgtggcaagagttgtaaataccgatgattatgtgactcgcacaagcatattttatcatgctggcagctctagattattaactgttggtaatccatattttagggttcctgcaggtggtggcaataagcaggatattcctaaggtttctgcataccaatatagagtatttagggtgcagttacctgacccaaataaatttggtttacctgatactagtatttataatcctgaaacacaacgtttagtgtgggcctgtgctggagtggaaattggccgtggtcagcctttaggtgttggccttagtgggcatccattttataataaattagatgacactgaaagttcccatgccgccacgtctaatgtttctgaggacgttagggacaatgtgtctgtagattataagcagacacagttatgtattttgggctgtgcccctgctattggggaacactgggctaaaggcactgcttgtaaatcgcgtcctttatcacagggcgattgcccccctttagaacttaaaaacacagttttggaagatggtgatatggtagatactggatatggtgccatggactttagtacattgcaagatactaaatgtgaggtaccattggatatttgtcagtctatttgtaaatatcctgattatttacaaatgtctgcagatccttatggggattccatgtttttttgcttacggcgtgagcagctttttgctaggcatttttggaatagagcaggtactatgggtgacactgtgcctcaatccttatatattaaaggcacaggtatgcgtgcttcacctggcagctgtgtgtattctccctctccaagtggctctattgttacctctgactcccagttgtttaataaaccatattggttacataaggcacagggtcataacaatggtgtttgctggcataatcaattatttgttactgtggtagataccactcgcagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctatattcatagtatgaatagcagtattttagaggattggaactttggtgttccccccccgccaactactagtttggtggatacatatcgttttgtacaatctgttgctattacctgtcaaaaggatgctgcaccggctgaaaataaggatccctatgataagttaaagttttggaatgtggatttaaaggaaagttttctttagacttagatcaatatccccttggacgtaaatttttggttcaggctgga

SEQ ID NO: 2 - protein, 500 aa, Human papillomavirus type 18
MALWRPSDNTVYLPPPSVARVVNTDDYVTRTSIFYHAGSSRLLTVGNPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKR

SEQ ID NO: 3 - DNA, 189 bp, Human papillomavirus type 18
atgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtaa

SEQ ID NO: 4 - protein, 105 aa, Human papillomavirus type 18
MHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQ

SEQ ID NO: 5 - DNA, 36 bp, Artificial Sequence
  Description of Artificial Sequence Primer
accagactcgagatggctttgtggcggcctagtgac

SEQ ID NO: 6 - DNA, 42 bp, Artificial Sequence
  Description of Artificial Sequence Primer
atagccaagcttaatgatatcctgaaccaaaaatttacgtcc

SEQ ID NO: 7 - DNA, 36 bp, Artificial Sequence
  Description of Artificial Sequence Primer
ggccatgatatcatgcatggacctaaggcaacattg

SEQ ID NO: 8 - DNA, 36 bp, Artificial Sequence
  Description of Artificial Sequence Primer
ggccatgatatctcgtcgggctggtaaatgttgatg

SEQ ID NO: 9 - DNA, 35 bp, Artificial Sequence
  Description of Artificial Sequence Primer
ggccatgatatctgtgtgacgttgtggttcggctc

SEQ ID NO: 10 - DNA, 36 bp, Artificial Sequence
  Description of Artificial Sequence Primer
cgccgcctcgagagatctatggctttgtggcggcct

SEQ ID NO: 11 - DNA, 42 bp, Artificial Sequence
  Description of Artificial Sequence Primer
ccggaattcccaccaatgcattccagcctgaaccaaaaattt

SEQ ID NO: 12 - DNA, 33 bp, Artificial Sequence
  Description of Artificial Sequence Primer
cgcggatccatgcatggacctaaggcaacattg

SEQ ID NO: 13 - DNA, 33 bp, Artificial Sequence
  Description of Artificial Sequence Primer
ccggaattcttattcggctcgtcgggctggtaa

SEQ ID NO: 14 - DNA, 33 bp, Artificial Sequence
  Description of Artificial Sequence Primer
ccggaattcttattgtggttcggctcgtcgggc

SEQ ID NO: 15 - DNA, 33 bp, Artificial Sequence
  Description of Artificial Sequence Primer
ccggaattcttacaacattgtgtgacgttgtgg

SEQ ID NO: 16 - DNA, 33 bp, Artificial Sequence
  Description of Artificial Sequence Primer
ccggaattcttacatacacaacattgtgtgacg

SEQ ID NO: 17 - DNA, 45 bp, Artificial Sequence
  Description of Artificial Sequence Primer
ttgcaatgttgccttaggtccatgtccagcctgaaccaaaaattt

SEQ ID NO: 18 - DNA, 45 bp, Artificial Sequence
  Description of Artificial Sequence Primer
gtcttgcaatgttgccttaggtcctccagcctgaaccaaaaattt

SEQ ID NO: 19 - DNA, 45 bp, Artificial Sequence
  Description of Artificial Sequence Primer
aaatttttggttcaggctggacatggacctaaggcaacattgcaa

SEQ ID NO: 20 - DNA, 45 bp, Artificial Sequence
  Description of Artificial Sequence Primer
aaatttttggttcaggctggaggacctaaggcaacattgcaagac

SEQ ID NO: 21 - protein, 20 aa, Human papillomavirus type 18
MALWRPSDNTVYLPPPSVAR

SEQ ID NO: 22 - protein, 20 aa, Human papillomavirus type 18
YLPPPSVARVVNTDDYVTRT

SEQ ID NO: 23 - protein, 20 aa, Human papillomavirus type 18
NTDDYVTRTSIFYHAGSSRL

SEQ ID NO: 24 - protein, 20 aa, Human papillomavirus type 18
FYHAGSSRLLTVGNPYFRVP

SEQ ID NO: 25 - protein, 20 aa, Human papillomavirus type 18
VGNPYFRVPAGGGNKQDIPK

SEQ ID NO: 26 - protein, 20 aa, Human papillomavirus type 18
GGNKQDIPKVSAYQYRVFRV

SEQ ID NO: 27 - protein, 20 aa, Human papillomavirus type 18
AYQYRVFRVQLPDPNKFGLP

SEQ ID NO: 28 - protein, 20 aa, Human papillomavirus type 18
PDPNKFGLPDTSIYNPETQR

SEQ ID NO: 29 - protein, 20 aa, Human papillomavirus type 18
SIYNPETQRLVWACAGVEIG

SEQ ID NO: 30 - protein, 20 aa, Human papillomavirus type 18
WACAGVEIGRGQPLGVGLSG

SEQ ID NO: 31 - protein, 20 aa, Human papillomavirus type 18
QPLGVGLSGHPFYNKLDDTE

SEQ ID NO: 32 - protein, 20 aa, Human papillomavirus type 18
FYNKLDDTESSHAATSNVSE

SEQ ID NO: 33 - protein, 20 aa, Human papillomavirus type 18
HAATSNVSEDVRDNVSVDYK

SEQ ID NO: 34 - protein, 20 aa, Human papillomavirus type 18
RDNVSVDYKQTQLCILGCAP

SEQ ID NO: 35 - protein, 20 aa, Human papillomavirus type 18
QLCILGCAPAIGEHWAKGTA

SEQ ID NO: 36 - protein, 20 aa, Human papillomavirus type 18
GEHWAKGTACKSRPLSQGDC

SEQ ID NO: 37 - protein, 20 aa, Human papillomavirus type 18
SRPLSQGDCPPLELKNTVLE

SEQ ID NO: 38 - protein, 20 aa, Human papillomavirus type 18
LELKNTVLEDGDMVDTGYGA

SEQ ID NO: 39 - protein, 20 aa, Human papillomavirus type 18
DMVDTGYGAMDFSTLQDTKC

SEQ ID NO: 40 - protein, 20 aa, Human papillomavirus type 18
FSTLQDTKCEVPLDICQSIC

SEQ ID NO: 41 - protein, 20 aa, Human papillomavirus type 18
PLDICQSICKYPDYLQMSAD

SEQ ID NO: 42 - protein, 20 aa, Human papillomavirus type 18
PDYLQMSADPYGDSMFFCLR

SEQ ID NO: 43 - protein, 20 aa, Human papillomavirus type 18
GDSMFFCLRREQLFARHFWN

SEQ ID NO: 44 - protein, 20 aa, Human papillomavirus type 18
QLFARHFWNRAGTMGDTVPQ

SEQ ID NO: 45 - protein, 20 aa, Human papillomavirus type 18
GTMGDTVPQSLYIKGTGMRA

SEQ ID NO: 46 - protein, 20 aa, Human papillomavirus type 18
YIKGTGMRASPGSCVYSPSP

SEQ ID NO: 47 - protein, 20 aa, Human papillomavirus type 18
GSCVYSPSPSGSIVTSDSQL

SEQ ID NO: 48 - protein, 20 aa, Human papillomavirus type 18
SIVTSDSQLFNKPYWLHKAQ

SEQ ID NO: 49 - protein, 20 aa, Human papillomavirus type 18
KPYWLHKAQGHNNGVCWHNQ

SEQ ID NO: 50 - protein, 20 aa, Human papillomavirus type 18
NNGVCWHNQLFVTVVDTTRS

SEQ ID NO: 51 - protein, 20 aa, Human papillomavirus type 18
VTVVDTTRSTNLTICASTQS

SEQ ID NO: 52 - protein, 20 aa, Human papillomavirus type 18
LTICASTQSPVPGQYDATKF

SEQ ID NO: 53 - protein, 20 aa, Human papillomavirus type 18
PGQYDATKFKQYSRHVEEYD

SEQ ID NO: 54 - protein, 20 aa, Human papillomavirus type 18
YSRHVEEYDLQFIFQLCTIT

SEQ ID NO: 55 - protein, 20 aa, Human papillomavirus type 18
FIFQLCTITLTADVMSYIHS

SEQ ID NO: 56 - protein, 20 aa, Human papillomavirus type 18
ADVMSYIHSMNSSILEDWNF

SEQ ID NO: 57 - protein, 20 aa, Human papillomavirus type 18
SSILEDWNFGVPPPPTTSLV

SEQ ID NO: 58 - protein, 20 aa, Human papillomavirus type 18
PPPPTTSLVDTYRFVQSVAI

SEQ ID NO: 59 - protein, 20 aa, Human papillomavirus type 18
YRFVQSVAITCQKDAAPAEN

SEQ ID NO: 60 - protein, 20 aa, Human papillomavirus type 18
QKDAAPAENKDPYDKLKFWN

SEQ ID NO: 61 - protein, 20 aa, Human papillomavirus type 18
PYDKLKFWNVDLKEKFSLDL

SEQ ID NO: 62 - protein, 20 aa, Human papillomavirus type 18
LKEKFSLDLDQYPLGRKFLV

SEQ ID NO: 63 - protein, 20 aa, Human papillomavirus type 18
YPLGRKFLVQAGMHGPKATL

SEQ ID NO: 64 - protein, 20 aa, Human papillomavirus type 18
MHGPKATLQDIVLHLEPQNE

SEQ ID NO: 65 - protein, 20 aa, Human papillomavirus type 18
VLHLEPQNEIPVDLLCHEQL

SEQ ID NO: 66 - protein, 20 aa, Human papillomavirus type 18
VDLLCHEQLSDSEEENDEID

SEQ ID NO: 67 - protein, 20 aa, Human papillomavirus type 18
SEEENDEIDGVNHQHLPARR

SEQ ID NO: 68 - protein, 20 aa, Human papillomavirus type 18
NHQHLPARRAEPQRHTMLCM

SEQ ID NO: 69 - protein, 20 aa, Human papillomavirus type 18
PQRHTMLCMCCKCEARIKLV

SEQ ID NO: 70 - protein, 20 aa, Human papillomavirus type 18
KCEARIKLVVESSADDLRAF

SEQ ID NO: 71 - protein, 20 aa, Human papillomavirus type 18
SSADDLRAFQQLFLNTLSFV

SEQ ID NO: 72 - protein, 17 aa, Human papillomavirus type 18
LFLNTLSFVCPWCASQQ